(19) United States
(12) Patent Application Publication
(10) Pub. No.: US 2004/0020495 A1
(43) Pub. Date: Feb. 5, 2004

(54) Miniature condom inside hollowed out hazelnut sealed with wood filler

(76) Inventor: Kevin G. Davis, (US)

Correspondence Address: KEVIN DAVIS, 232 BRIDGE LANDING COURT, VIRGINIA BEACH, VA, 23454, US

(21) Appl. No.: 10/211,885

(22) Filed: Aug. 2, 2002

(51) Int. Cl. A61F 6/04

(52) U.S. Cl. 128/844; 446/75; 446/76; 206/457

(57) ABSTRACT

A practical joke device in the embodiment of a hazelnut which when broken open with object of user choice, will create an illusion of displaying the interior contents of a miniature condom.

### CROSS-REFERENCE TO RELATED APPLICATIONS

Not Applicable

### FEDERALLY SPONSORED RESEARCH

Not Applicable

### SEQUENCE LISTING OR PROGRAM

Not Applicable

### FIELD OF INVENTION

This invention relates to gag or novelty devices that are used for practical jokes, but more particularly relates to such a device that when broken open, reveals the surprise of a small finger cot which resembles a miniature condom. Furthermore, when the device is in intact form, the device gives the illusion that is an ordinary hazelnut.

### BACKGROUND OF INVENTION

Novelty devices or gag devices have been known and used throughout history, as the general public always enjoys a good laugh. Such devices are commonly sold in stores and/or they are often seen in TV show, such as the gold show “CANDID CAMERA”. Furthermore, some of these devices have become extremely well known, such as the famous “WHOOPIE CUSHION”, or the “PET ROCK”, etc.

There are hundreds of novelty items sold in stores everywhere each year and there are just as many patents on various novelty items. However, nowhere in prior art did this applicant find a novelty item such as taught within the present application, wherein I provide a unique novelty item that creates an illusion of an intact hazelnut, that when opened, reveals what appears to be a miniature condom as the contents within.

### SUMMARY OF THE INVENTION

It is therefore an object of the present invention to provide a unique hollowed out hazelnut that stores a miniature condom within and is then sealed with wood filler novelty item that may be used as a gag device for humor purposes.

Other objects and advantages will be seen when taken into consideration with the following specification and drawings.

### BRIEF DESCRIPTION OF THE DRAWINGS

FIG. 1 is a substantial perspective overview of the preferred embodiment for the present invention.

FIG. 2 is a substantial perspective overview of the present invention when broken open to expose the interior contents of a finger cot creating the illusion of a miniature condom being the original contents within the hazelnut.

### DETAILED DESCRIPTIONS OF THE DRAWINGS

Referring now in detail to the drawings where in like characters refer to like elements throughout the various views.

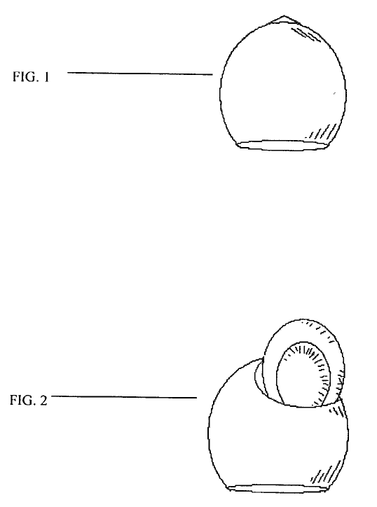

Within FIG. 1 (arrow 1) substantially represents an overview of the present invention which is a hollowed out hazelnut filled with a miniature condom and sealed with wood filler novelty device that is used for practical joke purposes. The device is unique and novel as it may be used to create an illusion that the device when broken open will reveal what appears to be a miniature condom as the true and original contents from within the hazelnut, this is unlike any other prior art device the applicant is aware of.

Referring now to FIG. 1 wherein we show the preferred exterior embodiment for the present invention, with exterior appearance being that of a hazelnut (arrow 1) novelty device comprising a hazelnut shell exterior, a miniature condom interior and a hole at the base of the exterior that has been sealed with wood filler through which the miniature condom was inserted. It is to be understood that the device is so constructed as to resemble an ordinary hazelnut.

Referring now to FIG. 2 where in we show the preferred interior embodiment for the present invention, with the interior creating an illusion that the device, when broken open, reveals what appears to be a miniature condom (arrow 2) as the true and original contents within.

I further provide a method of using a hollowed out hazelnut filled with a miniature condom and sealed with wood filler novelty device comprising the following steps:

a. locating a hard surface of user choice

b. grasping the hollowed out hazelnut filled with a miniature condom and sealed with wood filler novelty device

c. positioning the hollowed out hazelnut filled with a miniature condom and sealed with wood filler novelty device on that hard surface

d. grasping an object of considerable weight and solidity, and

e. using that object to shatter the hollowed out hazelnut filled with a miniature condom and sealed with wood filler novelty device, to expose the interior contents.

Although the invention has been herein shown and described in what is conceived to be the most practical and preferred embodiment, it is recognized that departures may be made therefrom within the scope and spirit of the invention, which is not limited to the details disclosed herein but is to be accorded full scope of the claims so as to embrace any and all equivalent devices and apparatus's.

Having described the invention what we claim as new and wish to secure by Letters Patent is:

1. A miniature condom inside a hollowed hazelnut sealed with wood filler novelty device comprising: hazelnut shell exterior, a miniature condom interior and a hole at the base of the exterior that has been sealed with wood filler through which the miniature condom was inserted, whereby:
when said device is broken open, reveals what appears to be a miniature condom as the true and original contents within.
